(19) United States
(12) Patent Application Publication
(10) Pub. No.: US 2017/0347918 A1
(43) Pub. Date: Dec. 7, 2017

(54) BREATH CAPTURE DEVICE

(71) Applicant: Pulmostics Limited, Dublin (IE)

(72) Inventor: William C. Arsenault, Newbury Park, CA (US)

(21) Appl. No.: 15/171,899

(22) Filed: Jun. 2, 2016

(51) Int. Cl. A61B 5/097; A61B 5/00; A61M 39/24

(52) U.S. Cl. CPC A61B 5/097; A61M 39/24; A61B 5/682; A61M 2039/242

(57) ABSTRACT

A breath capturing device including a body having an internal reservoir, an inlet, a first outlet, and a second outlet. A first pathway extends between the inlet and the reservoir, a second pathway extends between the reservoir and the first outlet, and a third pathway extends between the reservoir and the second outlet. A first valve is positioned within the first pathway and is configured to restrict flow therealong to flow from the inlet to the reservoir above a first pressure. A second valve is positioned within the second pathway and is configured to restrict flow therealong to flow from the reservoir to the first outlet above a second pressure greater than the first pressure. A third valve is positioned within the third pathway and is selectively transitional between an open and closed configurations to selectively retrieve fluid from the reservoir.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

### 1. Technical Field

The present disclosure relates generally to a fluid capturing device, and more specifically to a device sized and configured to capture at least a portion of an exhalation phase of a user's breath for diagnostic analysis.

### 2. Description of the Related Art

Medical treatment and care generally require knowledge of an underlying medical condition. A determination of the underlying medical condition may be obtained by various diagnostic tests or evaluations. Along these lines, a rapidly emerging diagnostic tool in the medical community is breath analysis, which is a desirable, non-invasive, relatively low cost tool allowing medical professionals to quickly diagnose a number of different medical conditions. For instance, breath analysis may be used to detect asthma, blood alcohol, lung cancer, breast cancer, diabetes, cystic fibrosis, malaria, Parkinson's Disease, leukemia, organ transplant rejection, radiation exposure, preeclampsia of pregnancy, liver disease, kidney disease, heart disease, sepsis, pneumonia, tuberculosis, among others.

Many breath analysis systems identify characteristic biomarkers and analyze signal patterns with high sensitivity and accuracy to reach a medical diagnosis. In many cases, the diagnosis can be made in just a few minutes on site, rather than sending a test sample to an off-site laboratory, as is typically required of a blood sample or other samples of bodily fluids. The on-site testing of the breath sample also mitigates potential sample degrading and contamination during sample transport.

Although the advantages of breath sampling are well-known, certain challenges remain in connection with obtaining the breath sample. For instance, it is important to collect the breath sample such that the sample is free from environmental contaminants. Furthermore, it is also desirable to collect alveolar fluid from the patient which is more representative of biomarkers coming from the patient's body and not environmental volatile organic compounds (VOCs) that the patient may be inhaling from the ambient environment, such as cleaning compounds or other environmental VOCs.

Accordingly, there is a need in the art for a device capable of easily and effectively collecting a breath sample for subsequent diagnostic testing and analysis. Various aspects of the present disclosure address this particular need, as will be discussed in more detail below.

### BRIEF SUMMARY

In accordance with one embodiment of the present disclosure, there is provided a device and related method for capturing a user's breath for diagnostic analysis and testing, preferably the last 25-35% of the exhalation phase, which is commonly associated with the user's alveolar breath.

According to one embodiment, the breath capturing device includes a body having an internal reservoir, an inlet, a first outlet, and a second outlet. A first fluid pathway extends between the inlet and the internal reservoir, a second fluid pathway extends between the internal reservoir and the first outlet, and a third fluid pathway extends between the internal reservoir and the second outlet. A first valve is positioned within the first fluid pathway and is configured to restrict fluid flow along the first fluid pathway to fluid flow from the inlet to the internal reservoir above a first fluid pressure. A second valve is positioned within the second fluid pathway and is configured to restrict fluid flow along the second fluid pathway to fluid flow from the internal reservoir to the first outlet above a second fluid pressure greater than the first fluid pressure. A third valve is positioned within the third fluid pathway and is selectively transitional between an open configuration, wherein fluid flows along the third fluid pathway, and a closed configuration, wherein fluid is restricted from flowing along the third fluid pathway.

The body may include a central body, a first end body engageable with the central body via snap-fit engagement, and a second end body engageable with the central body via snap-fit engagement. The first end body may include a mouthpiece adapted to enable a mouth of a user to breathe into the device. A first gasket may extend between the central body and the first end body to create a fluid tight seal therebetween, and a second gasket may extend between the central body and the second end body to create a fluid tight seal therebetween. A bladder may be coupled to the first end body and the second end body, with the bladder at least partially defining the internal reservoir.

The first valve may be transitional between an open configuration, wherein fluid flows along the first fluid pathway, and a closed configuration, wherein fluid flow is restricted along the first fluid pathway, with the first valve being biased towards the closed position by a first biasing force. The first valve may be configured to transition to the open position when fluid entering the first valve is above the first fluid pressure to overcome the first biasing force.

The second valve may be transitional between an open configuration, wherein fluid flows along the second fluid pathway, and a closed configuration, wherein fluid flow is restricted along the second fluid pathway, with the second valve being biased towards the closed position by a second biasing force. The second valve may be configured to transition to the open position when fluid entering the second valve is above the second fluid pressure to overcome the second biasing force.

The first fluid pressure and the second fluid pressure may be associated with respective magnitudes to enable a portion of an expiration phase by a human and directed into the inlet to be captured within the internal reservoir.

According to another embodiment, there is provided a pressure actuated fluid capturing device comprising a body having an internal reservoir. An inlet valve is coupled to the body and is in fluid communication with the internal reservoir, with the inlet valve being biased toward a closed position wherein fluid is restricted from flowing into the internal reservoir through the inlet valve. The inlet valve transitions to an open position to enable fluid flow into the internal reservoir through the inlet valve in response to fluid pressure applied thereto exceeding a first pressure threshold. An outlet valve is coupled to the body and is in fluid communication with the internal reservoir. The outlet valve is biased toward a closed position wherein fluid is restricted from flowing out of the internal reservoir through outlet valve. The outlet valve transitions to an open position to enable fluid flow out of the internal reservoir through the outlet valve in response to fluid pressure applied thereto exceeding a second pressure threshold greater than the first pressure threshold. The first and second pressure thresholds are associated with respective fluid pressures to enable a portion of an expiration phase by a human and directed into the inlet valve to be captured within the internal reservoir.

According to yet another embodiment, there is provided a method of capturing a portion of a user's breath. The method includes: receiving breath exhaled from a user in a breath capturing device; capturing a first portion of the breath exhaled by the user within the breath capturing device such that the fluid pressure within the breath capturing device increases; venting at least a portion of the captured breath from the breath capturing device when the fluid pressure within the breath capturing device exceeds a prescribed pressure threshold; and capturing a second portion of the breath exhaled by the user and subsequent to the venting step in response to the fluid pressure within the breath capturing device falling below the prescribed pressure threshold.

All steps of the method may be performed during a continuous exhalation phase by the user. The first and second portions of the breath may be exhaled by the user during a continuous exhalation phase.

The breath capturing device may include an exhaust port which remains closed while capturing the first portion of the breath exhaled by the user, and which opens during the venting step.

The venting step may include transitioning a vent valve from a closed position to an open position in response to fluid pressure within the breath capturing device exceeding a prescribed vent threshold. The step of capturing the second portion of the breath may include transitioning the vent valve from the open position to the closed position in response to the fluid pressure within the breath capturing device falling below the prescribed vent threshold.

The present disclosure will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which:

FIG. 1 is an upper perspective view of one embodiment of a breath capturing device;

FIG. 2 is an exploded perspective view of the breath capturing device depicted in FIG. 1;

FIG. 3 is a cross sectional view of the breath capturing device during a first exhalation phase;

FIG. 3A is an enlarged, sectional view depicting cooperative engagement between a first end body and a central body;

FIG. 4 is a cross sectional view of the breath capturing device during a second exhalation phase;

FIG. 5 is a cross sectional view of the breath capturing device during a third exhalation phase;

FIG. 6 is a schematic diagram illustrating signal communication between the breath capturing device and a breath testing device;

FIG. 7 is an upper perspective view of the breath capturing device and the breath testing device, prior to the breath capturing device being cooperatively engaged with the breath testing device;

FIG. 8 is an upper perspective view of the breath capturing device cooperatively engaged with the breath testing device;

FIG. 9 is a cross sectional view of the breath capturing device cooperatively engaged with the breath testing device to enable extraction of the collected breath sample; and

FIG. 10 is a pressure curve approximating a typical human breathing cycle.

Common reference numerals are used throughout the drawings and the detailed description to indicate the same elements.

### DETAILED DESCRIPTION

The detailed description set forth below in connection with the appended drawings is intended as a description of certain embodiments of a breath capturing device and related method of use, and is not intended to represent the only forms that may be developed or utilized. The description sets forth the various structure and/or functions in connection with the illustrated embodiments, but it is to be understood, however, that the same or equivalent structure and/or functions may be accomplished by different embodiments that are also intended to be encompassed within the scope of the present disclosure. It is further understood that the use of relational terms such as first and second, and the like are used solely to distinguish one entity from another without necessarily requiring or implying any actual such relationship or order between such entities.

Various aspects of the present disclosure are directed toward capturing a user's breath within a device to enable medical testing of the breath captured within the device. Certain embodiments are specifically directed toward capturing only a portion of the user's exhalation phase of the user's breath, such as approximately the last one-third of the user's exhalation phase, which corresponds to the aveolar breath.

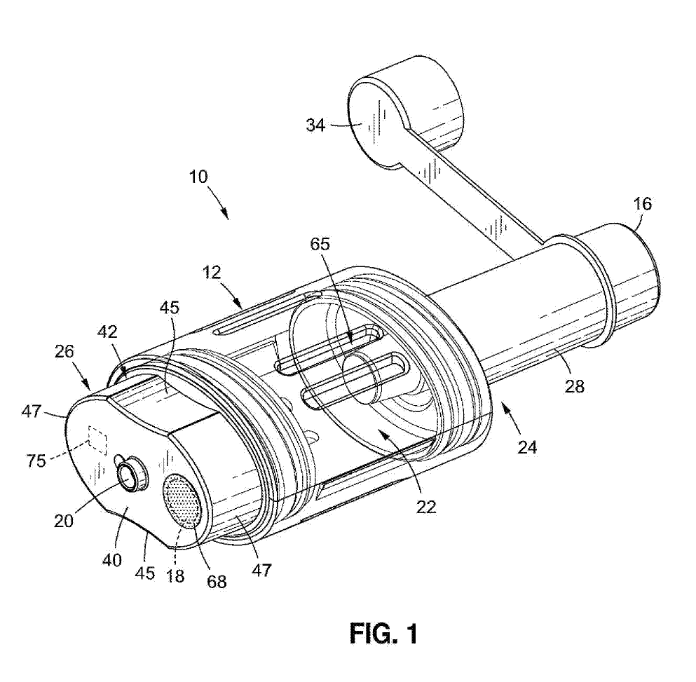

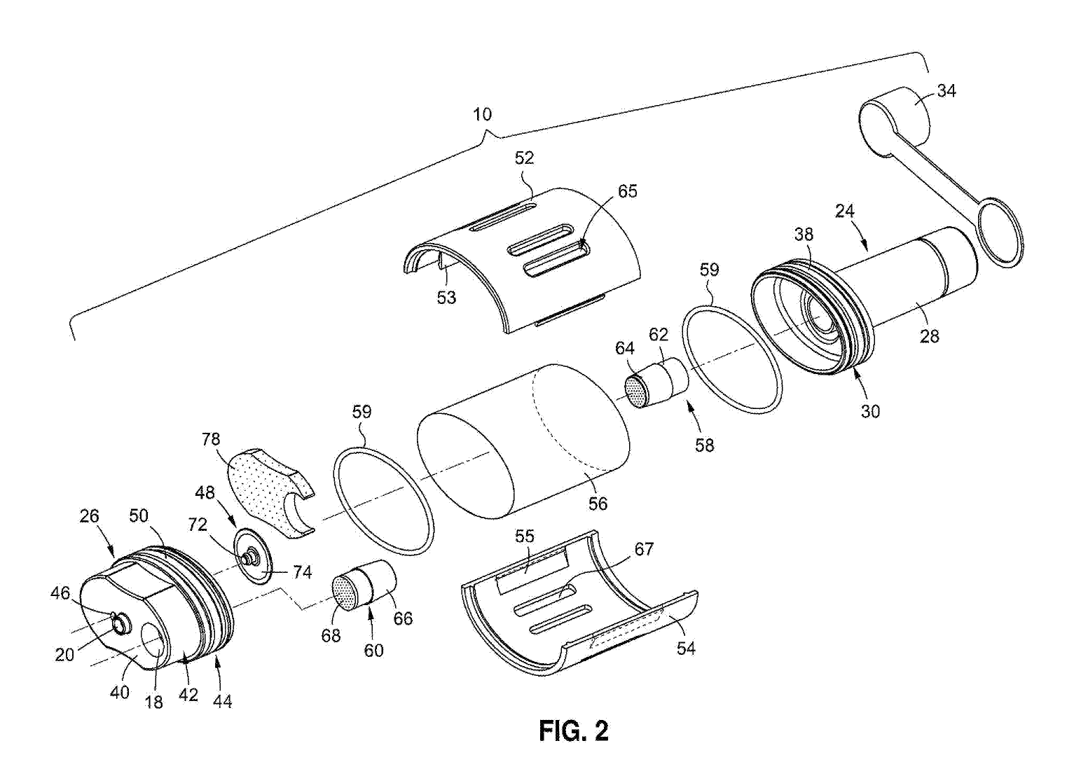

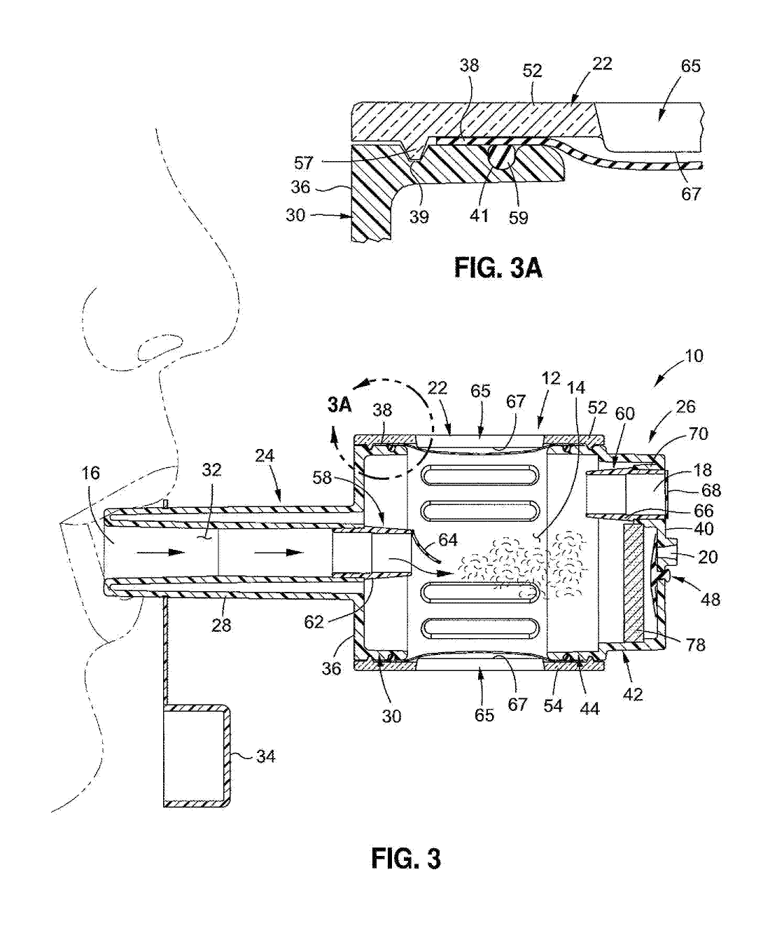

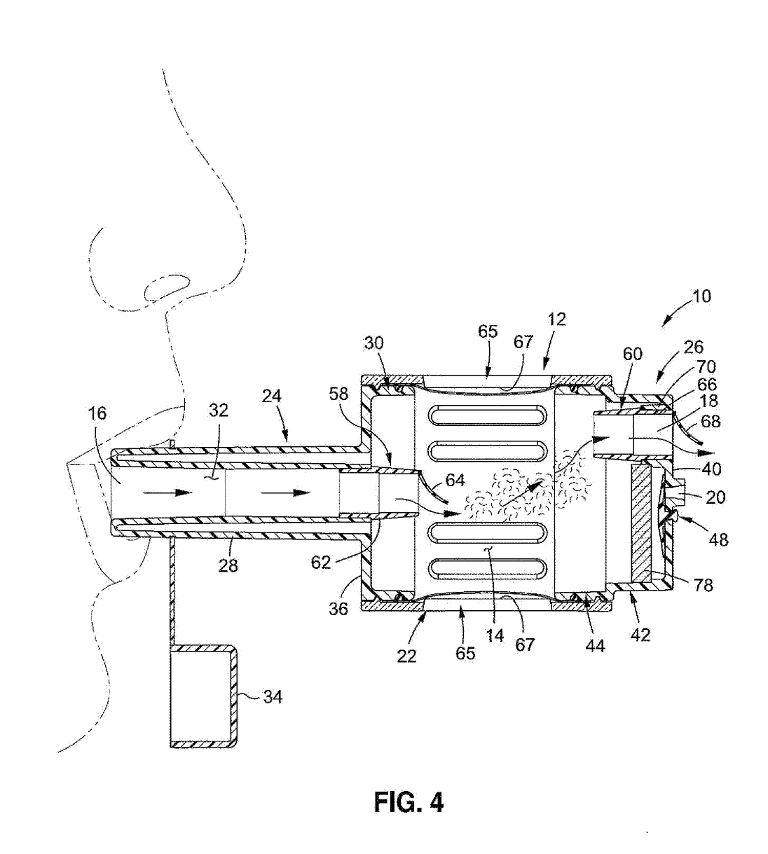

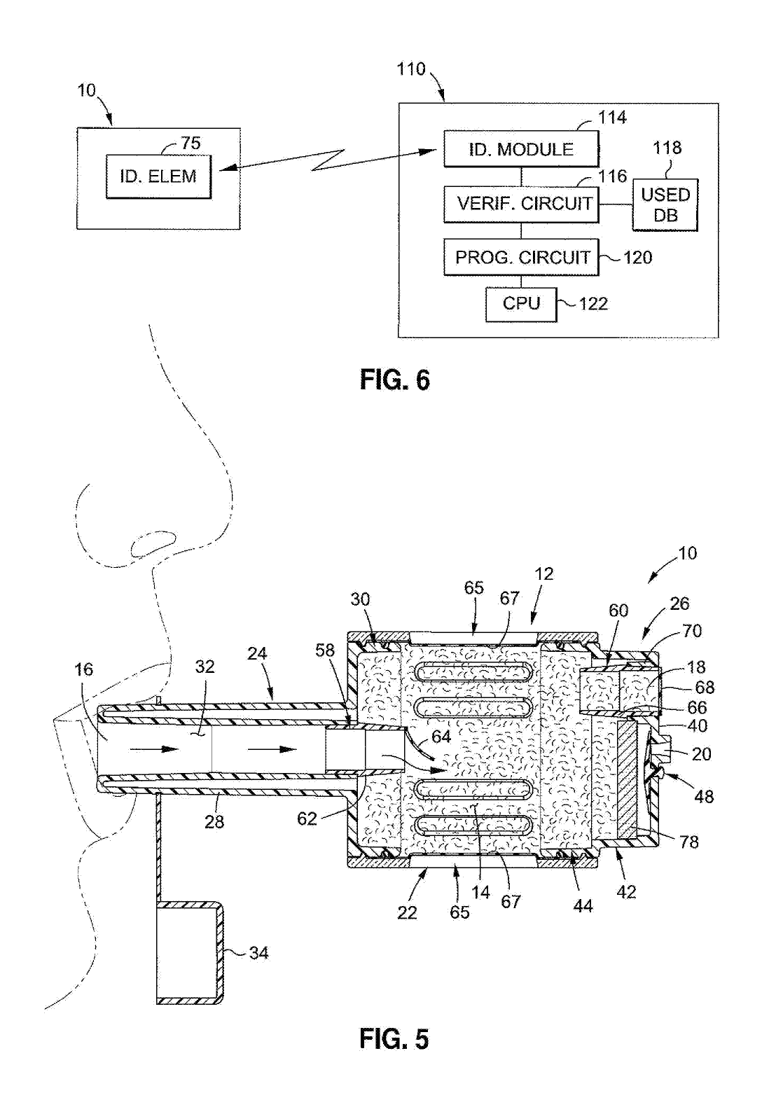

According to one embodiment, and referring now to the drawings, a breath capturing device 10 includes a body 12 having an internal reservoir 14, an inlet 16, a first outlet 18, and a second outlet 20 (e.g., an extraction port). FIG. 1 shows the device 10 in an assembled configuration, while FIG. 2 shows the device 10 in an exploded configuration. According to one embodiment, the body 12 is a multi-piece assembly generally including a central body 22, a first end body 24 and a second end body 26. The first and second end bodies 24, 26 may be formed from Polyether Ether Ketone (PEEK) or other materials known in the art. The first end body 24 includes a mouthpiece 28 and a first connector 30 coupled to the mouthpiece 28. Preferably, the mouthpiece 28 and the first connector 30 are integrally formed with each other. The mouthpiece 28 is tubular and includes an outer surface and an inner surface defining an opening 32 extending axially through the first end body 24. The mouthpiece 28 is sized and configured to allow a user to blow or exhale into the device 10 through the mouthpiece 28 with the user's mouth. An end cap 34 may be included to cover an end portion of the mouthpiece 28 during periods of nonuse. The end cap 34 may be placed over the mouthpiece 28 to prevent ambient air from being drawn into the device 10 while a collected breath sample is retrieved from the device 10. Furthermore, the mouthpiece 28 may be covered by the end cap to allow the device 10 to be moved without ambient air flowing into the device 10. The first connector 30 extends radially outward from the mouthpiece 28 and includes an end wall 36 and a first connecting surface 38 (see FIGS. 2, 3) extending axially from the end wall 36. The first connecting surface 38 includes a pair of circumferential grooves 39, 41 spaced apart from each other, with the grooves 39, 41 being adapted to facilitate engagement between the first end body 24 and the central body 22, as will be described in more detail below.

The second end body 26 includes an end wall 40, an extension wall 42 and a second connector 44. The end wall 40 includes a pair of openings extending therethrough forming respective ones of the first and second outlets 18, 20 to facilitate selective fluid flow through the second end body 26, as will be described in more detail below. A third opening 46 (see FIG. 2) may be formed in the end wall 40 to provide a mounting structure for an umbrella valve 48. The extension wall 42 extends from a peripheral portion of the end wall 40 in an axial direction toward the second connector 44. The second connector 44 includes a second connector surface 50 including a pair of circumferential grooves spaced apart from each other, with the grooves being adapted to facilitate engagement between the second end body 26 and the central body 22, as will be described in more detail below. The pair of circumferential grooves formed in the second connector 44 are similar to the pair of circumferential grooves formed in the first connector 30.

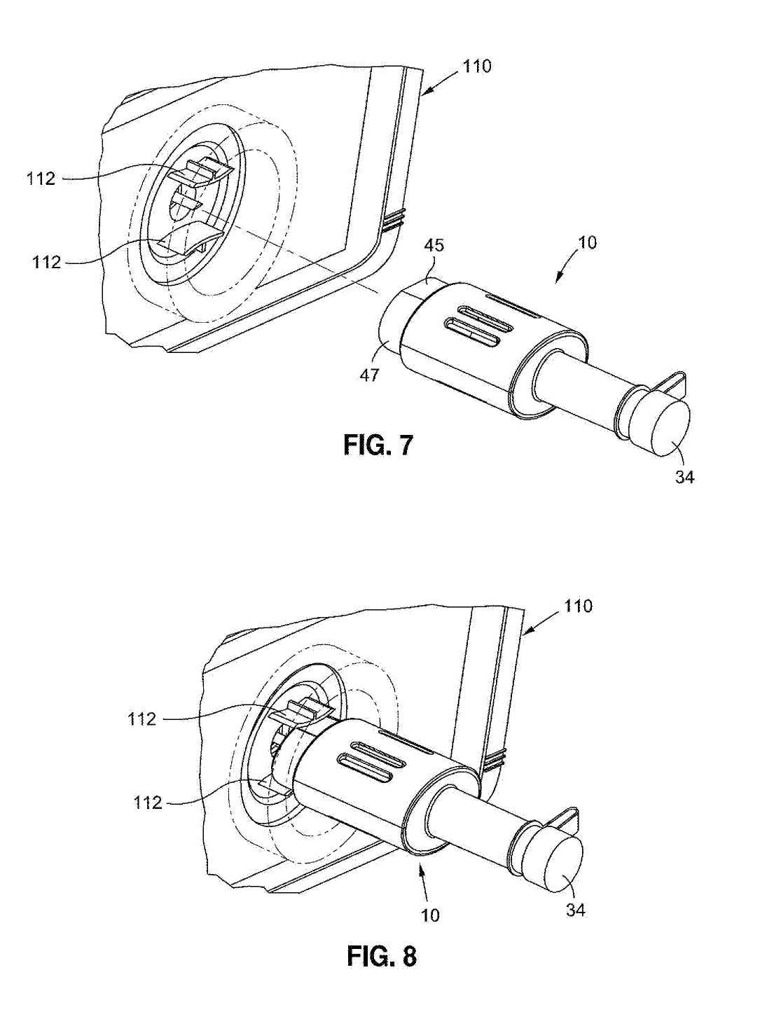

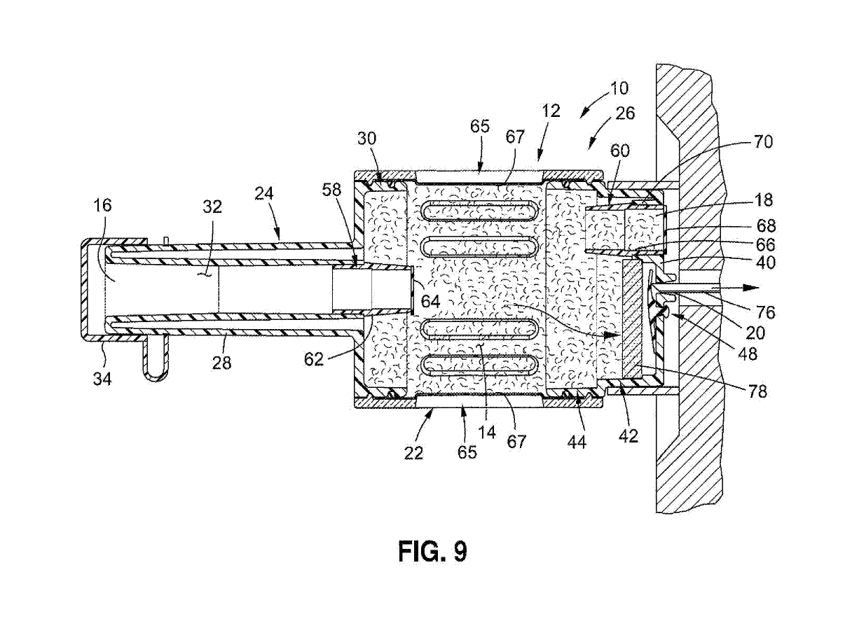

According to one embodiment, the second end body 26 is specifically configured to be complimentary in shape to a corresponding opening or mating structure formed on breath testing machine 110 (see FIGS. 7 and 8) to enable cooperative engagement between the second end body 26 and the breath testing machine 110 for purposes of transferring the breath sample from the capturing device 10 to the testing machine 110. As shown in the exemplary embodiment, the extension wall 42 of the second end body 26 includes a pair of opposed, convex, arcuate segments 45, which are separated by a pair of opposed arcuate, concave segments 47. The testing device 110 includes a pair of opposed tabs 112 which are complimentary to the pair of opposed concave segments 45 formed on the capturing device 10, such that when the capturing device 10 is inserted into the testing device 110, the complimentary arrangement between the tabs 112 and the concave surfaces 45 registers the capturing device 10 relative to the testing device 110. Of course, the scope of the present disclosure is not limited to the particular configuration of the second end body 26 shown and described herein and the corresponding structure on the breath testing device 110 depicted in the drawings. In this regard, the second end body 26 and the breath testing device 110 may include other configurations without departing from the spirit and scope of the present disclosure.

The central body 22 serves as a shell which extends at least partially over the internal reservoir 14 and between the first and second end bodies 24, 26. According to one embodiment, the central body 22 is a multi-piece assembly including a first half body 52 and a second half body 54. The first half body 52 includes a pair of tabs 53 which are insertable with corresponding recesses 55 formed on the second half body 54 to facilitate snap-fit engagement between the first and second half bodies 52, 54. The central body 22 includes a first end portion adapted to be cooperatively engageable with the first end body 24 and a second end portion adapted to be cooperatively engageable with the second end body 26. Referring now specifically to FIG. 3A, each end portion of the central body 22 includes a protrusion 57 adapted to be received within a corresponding circumferential groove 39 formed on one of the first and second end bodies 24, 26 to effectuate engagement between the central body 22 and the first and second end bodies 24, 26. Furthermore, a pair of gaskets or o-rings 59 are insertable within a corresponding groove 41 formed on the first and second end bodies 24, 26 to create fluid-tight seal between the central body 22 and the first and second end bodies 24, 26.

According to one embodiment, the outer diameter of the o-rings is slightly larger than the inner diameter of the central body 22, which is collectively defined by the first half body 24 and second half body 26 when the first half body 24 is engaged with the second half body 26. The larger diameter of the o-rings requires compression of the o-rings when the central body 22 is engaged with the first and second end bodies 24, 26. As can be seen in FIG. 3A, the top of the o-ring 59 is compressed due to the engagement between central body 22 and end body 30. The compression of the o-rings 59 results in a compression fit between the central body 22 and end bodies 24, 26. The compression fit makes the device 10 more resistant to disassembly when the device 10 is inadvertently dropped.

The central body 22 further includes at least one, and preferably a plurality of windows 65 formed therein, with each window 65 being recessed from an outer surface, and defining a window inner surface 67. The windows 65 may be elongate and define an ovular shape, a rectangular shape, or any shape known in the art. The windows 65 provide a visual indication as to the volume of the breath sample collected, as will be described in more detail below.

According to one embodiment, the central body 22, first end body 24 and second end body 26 are collectively configured to enable snap-fit engagement therebetween, and thus, additional mechanical fasteners, adhesives, etc., may not be required, which may simplify the overall manufacture and use of the device 10. Such ease in manufacture may lower the overall production cost, thereby making certain embodiments of the device 10 disposable or intended for one-time use. As such, the user may be assured that when using the device 10, the collected breath sample may not be contaminated or altered by a previous breath sample. The use of the gaskets or o-rings 59 creates the fluid-tight engagement between the end bodies 24, 26 and the central body 22 without the use of adhesives. Furthermore, if adhesives are not used in the device 10, the breath sample is not tainted via outgassing of vapors or other contaminants, as may be associated with various adhesives. However, it is understood with suitable cleaning during manufacturing or by taking other protective measures known in the art, adhesives may be used in other embodiments of the device 10 without departing from the spirit and scope of the present disclosure.

A bladder 56 may be coupled to the first end body 24 and the second end body 26, with the bladder 56 at least partially defining the internal reservoir 14. In this regard, the bladder 56 may extend over the internal surface of the central body 22, with respective end portions of the bladder 56 being captured between the central body 22 and the first and second end bodies 24, 26. The bladder 56 transitions from a deflated configuration to an inflated configuration as the user exhales into the device 10. In the deflated configuration, a central portion of the bladder 56 is spaced from the inner window surfaces 67 of the central body 22. When the bladder 56 is completely inflated, the outer surface of the bladder 56 touches the inner window surfaces 67 of the central body 22, with such contact between the bladder 56 and the inner window surfaces 67 providing a visual indicator to the user that a known volume of breath sample has been collected, which may be critical for diagnostic testing of the collected breath. Furthermore, when the breath sample is subsequently retrieved from the device 10, at least a portion of the bladder 65 moves away from an inner window surface 67 to provide a visual indication that the breath sample has been extracted from the device 10. Thus, the movement of the bladder 65 relative to the inner window surfaces 67 of the windows 65 provides an indication to the user that a complete breath sample has been collected, as well as an indication that the breath sample has been subsequently extracted from the device 10. In order to enhance the visual indicating effect, the window 65 may be clear and the bladder 56 may be formed from a material having a color, such as red, which is easily viewable through the clear window 65. According to various embodiments, the volume of the internal reservoir 14 when the bladder 56 is completely inflated may range for 50 cc-200 cc, and in one particular embodiment, the volume is 100 cc. The bladder 56 may be formed from Fluorinated Ethylene Propylene (FEP) or other materials known by those skilled in the art. According to one embodiment, the bladder 56 defines a thickness in the range of 0.005-0.002, and more preferably, approximately 0.001, with the thickness being defined as the distance between the inner surface of the bladder 56 and the outer surface of the bladder 56.

The breath capturing device 10 is configured such that a first fluid pathway extends between the inlet 16 and the internal reservoir 14, a second fluid pathway extends between the internal reservoir 14 and the first outlet 18, and a third fluid pathway extends between the internal reservoir 14 and the second outlet 20. The device 10 includes a first valve 58 (e.g., an inlet valve) positioned within the first fluid pathway to restrict fluid flow therealong, a second valve 60 (e.g., an outlet valve) positioned within the second fluid pathway to restrict fluid flow therealong, and a third valve 48 (e.g., an extraction valve) positioned within the third fluid pathway to restrict fluid flow therealong. The first, second, and third valves 58, 60, 48 are configured to control fluid flow through the device 10 to capture a precise portion of the breath exhaled by the user, particularly, a desired portion of the user's exhalation phase of breathing. Along these lines, the first and second valves 58, 60 are specifically configured and adapted to open in response to prescribed fluid pressures being applied thereto. The third valve 48 is adapted to remain closed during the user's breathing, and is operative to enable extraction of the breath from the device 10 for testing.

According to one embodiment, the first valve 58 is a one-way valve configured to allow fluid flow along the first fluid pathway from the mouthpiece 28 into the internal reservoir 14. In this regard, the first valve 58 prevents fluid flow from the internal reservoir 14 to the mouthpiece 28. The first valve 58 includes a first valve body 62 and a first fluid restricting element 64 (e.g., a valve flapper) selectively transitional relative to the first valve body 62 between an open position and a closed position. In the exemplary embodiment, the first valve body 62 is sized and configured to engage with the first end body 24 to create a fluid tight fit therebetween. For instance, the first valve body 62 may frictionally engage with a surface of the first end body 24 to maintain the first valve body 62 in position relative to the first end body 24. The first valve body 62 defines a first valve body passageway that is in fluid communication with the first fluid passageway when the first valve 58 is mounted to the first end body 24. The first fluid restricting element 64 includes a flapper which extends across the first valve body passageway when in a closed position, and is capable of moving relative to the first valve body 62 to uncover at least a portion of the first valve body passageway to enable fluid communication through the first valve body 62. The flapper is biased toward the closed position by a biasing force associated with the first fluid pressure, wherein application of fluid pressure greater than or equal to the first fluid pressure overcomes the biasing force and transitions the flapper to the open position.

According to one embodiment, the second valve 60 is similar to the first valve 58, with the primary distinction being that the biasing force associated with the second valve 60 is greater than that biasing force associated with the first valve 58, and thus, an increased fluid pressure is required to overcome the biasing force of the second valve 60. More specifically, the second valve 60 is a one-way valve configured to allow fluid flow along the second fluid pathway from the internal reservoir 14 and through the first outlet 18. In this regard, the second valve 60 prevents fluid flow into the internal reservoir 14 from the first outlet 18. The second valve 60 includes a second valve body 66 and a second fluid restricting element 68 (e.g., a valve flapper) selectively transitional relative to the second valve body 66 between an open position and a closed position. In the exemplary embodiment, the second valve body 66 is sized and configured to engage with the second end body 26 to create a fluid tight fit therebetween. For instance, the second valve body 66 may frictionally engage with a surface of the second end body 26 to maintain the second valve body in position relative to the second end body. Along these lines, the second end body 26 may include an annular mounting wall 70 extending from the end wall 40, with the mounting wall 70 being sized and configured to frictionally engage with the second valve body 66 to create a fluid tight seal therebetween. The second valve body 66 defines a second valve body passageway that is in fluid communication with the second fluid passageway when the second valve 60 is mounted to the second end body 66. The second fluid restricting element 68 includes a flapper which extends across the second valve body passageway when in a closed position, and is capable of moving relative to the second valve body 66 to uncover at least a portion of the second valve body passageway to enable fluid communication through the second valve body 66. The flapper is biased toward the closed position by a biasing force associated with the second fluid pressure, wherein application of fluid pressure greater than or equal to the second fluid pressure overcomes the biasing force and transitions the flapper to the open position.

The first fluid pressure associated with the first valve 58 and the second fluid pressure associated with the second valve 60 correspond to respective magnitudes to enable a portion of an expiration phase by a human and directed into the inlet 16 to be captured within the internal reservoir 14. Furthermore, the pressure differential between the first fluid pressure and the second fluid pressure ensures the device 10 captures a known volume of the user's breath, such as approximately 100 cc. It is understood that the pressure differential may be different depending on the intended use of the device 10. For instance, the pressure differential may be larger for devices 10 intended for use by adults, and smaller for devices 10 intended for use by children.

The third valve 48 is operative to enable selective extraction of the breath captured within the device 10 through the second outlet 20. In this regard, the third valve 48 is designed to remain closed until the breath is extracted from the device 10, and thus, the third valve 48 is intended to remained closed while the user breaths into the device 10. According to one embodiment, the third valve 48 is an umbrella valve having a central mounting nub 72 and a radial body 74 extending radially outward from the central mounting nub 72. The umbrella valve may be formed from nitrile, or other materials known in the art. The central mounting nub 72 is sized and configured to extend through the third opening 46 formed on the end wall 40. It is also understood that in other embodiments, the end wall 40 may include a recess, instead of an opening, within which the central mounting nub 72 is mounted. The radial body 74 is sized and configured to extend over the extraction port 20 when the central mounting nub 72 is received within the third opening 46 so as to prevent fluid flow through the extraction port 20. The radial body 74 is biased toward the end wall 40 to effectively cover the extraction port 20 to prevent inadvertent fluid loss through the extraction port 20. The radial body 74 is capable of being flexed by an extraction tool 76 when the extraction tool 76 is inserted into the extraction port 20 to extract the breath from the device 10, as will be described in more detail below. By way of example, and not limitation, an exemplary extraction tool 76 is the Model 4600 zNose™ made by Electronic Sensor Technology, although it is contemplated that other extraction tools known by those skilled in the art may also be used without departing from the spirit and scope of the present disclosure. Although it may be preferable to extract the breath sample using a vacuum extraction device, it is also contemplated that certain embodiments of the device 10 may be configured to enable extraction of the breath sample via a pump mechanism.

The device 10 may also include a filter 78 in fluid communication with the third fluid pathway, such that fluid passes through the filter 78 as the fluid flows through the third fluid pathway. The filter 78 may be a moisture filter to reduce the amount of moisture in the tested breath sample, as moisture content in the breath sample when tested may be undesirable. It is contemplated that the filter 78 is not limited to a moisture filter, and that other filters known by those skilled in the art may be employed. The filter 78 may be captured between the mounting wall 70 and the extension wall 42 of the second end body 26.

According to one embodiment, the device 10 may be intended for one-time use, and thus, the device 10 may include an identification element 75 which may be read prior to use to ensure the device 10 has never been used before. The identification element 75 may include a radio-frequency identification (RFID) chip, a barcode, an alphanumeric code, a QR code, or other identifying elements known in the art. The breath testing device 110 may include an identification module 114 capable of reading the identification element 75 on the device 10 and a verification circuit 116 for verifying that the particular device 10 associated with the identification element 75 has never been used before. The identification module 114 may be complimentary to the identification element 75 used on the capturing device, and thus, may include a RFID reader, a barcode scanner, and optical character recognition module, etc. The verification circuit 116 may compare information associated with the identification element 75 with a database 118 of information associated with used devices 10. If the information matches, the breath testing device 110 may identify the capturing device 10 as having already been used, and will cease retrieving the breath sample from the device 10. If no match is found, the capturing device 10 may be identified as being a new device and one from which a breath sample may be retrieved. It is contemplated that the identification module 114 may be able to write information back onto the identification element 75, e.g., writing information back onto an RFID chip. For instance, the information written onto the identification element 75 may indicate that the device 10 has been used, which test was performed on the breath sample collected from the device 10, as well as information related to the patient, e.g., patient name, vitals, etc.

The identification element 75 may also be used for programming the breath testing device 110 to perform a specific breath testing procedure. Along these lines, the testing protocols associated with testing for one disease or condition may be different from the testing protocols associated with testing for another disease or conditions. Different VOCs or different levels of VOCs may be associated with different diseases or conditions, and thus, the breath testing device 110 may need to be specifically programmed to run a specific testing protocol. It is contemplated that the identification element 75 may be associated with a specific testing protocol, such that when the identification element 75 is read, scanned, or otherwise identified by the testing device 110, a programming circuit 120 within the testing device 110 will implement the specific testing protocols associated with the identified identification element 75. The implementation of the specific testing protocols may require communication with a CPU 122 or other onboard sensors or testing equipment.

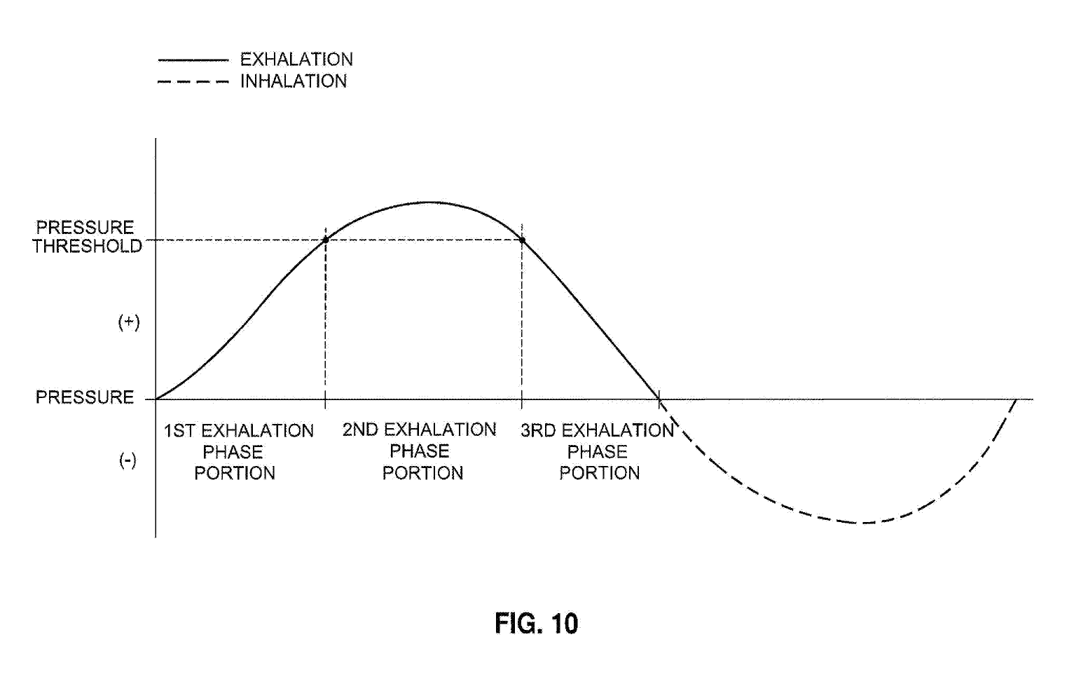

With the basic structure of the device 10 described above, the following discussion will focus on an exemplary use of the device 10. The device 10 is intended to be used to capture a portion of the breath associated with an exhalation phase of a user's breathing cycle. As can be seen in FIG. 10, a typical exhalation phase includes a variable pressure during the course of the exhalation phase, which is divided into three phase portions defined by a prescribed pressure threshold, P. The beginning portion of the exhalation phase having a pressure below the prescribed pressure threshold P is referred to as the first exhalation phase portion, the intermediate portion of the exhalation phase associated with a pressure above the prescribed pressure threshold P is referred to as the second exhalation phase, and the final portion of the exhalation phase associated with a pressure below the prescribed pressure threshold P is referred to as the third exhalation phase.

Use of the device 10 begins by placing the end of the mouthpiece within the user's mouth, and then the user breathing or exhaling into the device 10 at the beginning of the exhalation phase. As the user breaths, the breath exhaled from the user overcomes the first biasing force associated with the first valve 58 and enters the internal reservoir 14. While the pressure remains below the prescribed pressure threshold P, the second valve 60 remains closed, and the exhaled breath is retained within the internal reservoir 14.

As the user continues exhaling and the pressure crosses the pressure threshold P (e.g., the second exhalation phase portion), the second valve 60 opens to allow the exhaled breath within the device 10 to be exhausted through the first outlet 18. In this regard, when the pressure is above the pressure threshold P, a continuous fluid pathway is formed through the device 10 from the inlet 16 to the first outlet 18. In this regard, most of the fluid captured within the device 10 during the first and second exhalation phase portions may be exhausted or vented out of the device 10, and does not form the breath sample that is ultimately captured within the device 10.

As the user continues through the exhalation phase and the pressure falls below the prescribed pressure threshold P, the second valve 60 closes, and thus, the breath exhaled by the patient is captured within the internal reservoir 14. When the user completes the exhalation phase, the pressure applied to the device 10 by the user drops to zero, which causes the first valve 58 to transition to from the open position to the closed position. In this regard, the third portion of the exhalation phase of the user's breath is captured within the device 10, which results in the pressure within the internal reservoir 14 being elevated relative to the ambient environment.

In order to extract the breath sample from the device 10, the umbrella valve 48 is physically spaced from the second outlet 20 to uncover at least a portion of the second outlet 20. An extraction tool 76 may be inserted into the second outlet 20 to access the breath sample captured within the device 10. Since the breath sample is at an elevated pressure, the breath sample may flow out of the device 10 through the second outlet 20 and into the extraction tool 76. As an alternative, the extraction tool 76 may apply a vacuum to more rapidly extract the breath sample from the device 10, although, when a vacuum is applied, it is desirable to cover the mouthpiece 28, either with the end cap 34 or the user's finger/hand to prevent ambient air from being withdrawn into the extraction tool 76.

Once the breath sample is retrieved by the extraction tool 76, the breath sample may be analyzed to test for one or more medical conditions.

The particulars shown herein are by way of example only for purposes of illustrative discussion, and are not presented in the cause of providing what is believed to be most useful and readily understood description of the principles and conceptual aspects of the various embodiments of the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. A breath capturing device comprising:
a body having an internal reservoir, an inlet, a first outlet, and a second outlet, a first fluid pathway extending between the inlet and the internal reservoir, a second fluid pathway extending between the internal reservoir and the first outlet, and a third fluid pathway extending between the internal reservoir and the second outlet;
a first valve positioned within the first fluid pathway and configured to restrict fluid flow along the first fluid pathway to fluid flow from the inlet to the internal reservoir above a first fluid pressure;
a second valve positioned within the second fluid pathway and configured to restrict fluid flow along the second fluid pathway to fluid flow from the internal reservoir to the first outlet above a second fluid pressure greater than the first fluid pressure; and
a third valve positioned within the third fluid pathway and selectively transitional between an open configuration, wherein fluid flows along the third fluid pathway, and a closed configuration, wherein fluid is restricted from flowing along the third fluid pathway.

2. The breath capturing device as recited in claim 1, wherein the body includes:
a central body;
a first end body engageable with the central body via snap-fit engagement; and
a second end body engageable with the central body via snap-fit engagement.

3. The breath capturing device as recited in claim 2, further comprising a first gasket extending between the central body and the first end body to create a fluid tight seal therebetween, and a second gasket extending between the central body and the second end body to create a fluid tight seal therebetween.

4. The breath capturing device as recited in claim 2, further comprising a bladder coupled to the first end body and the second end body, the bladder at least partially defining the internal reservoir and being adapted to contact a prescribed surface on the body when a prescribed volume of fluid is received within the bladder, the bladder and the body being configured such that the contact between the bladder and the prescribed surface on the body provides a visual indication to a user.

5. The breath capturing device as recited in claim 2, wherein the first end body includes a mouthpiece adapted to enable a mouth of a user to breath into the device.

6. The breath capturing device as recited in claim 1, wherein the first valve is transitional between an open configuration, wherein fluid flows along the first fluid pathway, and a closed configuration, wherein fluid flow is restricted along the first fluid pathway, the first valve being biased towards the closed position by a first biasing force, the first valve being configured to transition to the open position when fluid entering the first valve is above the first fluid pressure to overcome the first biasing force.

7. The breath capturing device as recited in claim 1, wherein the second valve is transitional between an open configuration, wherein fluid flows along the second fluid pathway, and a closed configuration, wherein fluid flow is restricted along the second fluid pathway, the second valve being biased towards the closed position by a second biasing force, the second valve being configured to transition to the open position when fluid entering the second valve is above the second fluid pressure to overcome the second biasing force.

8. The breath capturing device as recited in claim 1, wherein the first fluid pressure and the second fluid pressure are associated with respective magnitudes to enable a portion of an expiration phase by a human and directed into the inlet to be captured within the internal reservoir.

9. A pressure actuated fluid capturing device comprising:
a body having an internal reservoir,
an inlet valve coupled to the body and in fluid communication with the internal reservoir, the inlet valve being biased toward a closed position wherein fluid is restricted from flowing into the internal reservoir through the inlet valve, the inlet valve transitioning to an open position to enable fluid flow into the internal reservoir through the inlet valve in response to fluid pressure applied thereto exceeding a first pressure threshold; and
an outlet valve coupled to the body and in fluid communication with the internal reservoir, the outlet valve being biased toward a closed position wherein fluid is restricted from flowing out of the internal reservoir through outlet valve, the outlet valve transitioning to an open position to enable fluid flow out of the internal reservoir through the outlet valve in response to fluid pressure applied thereto exceeding a second pressure threshold greater than the first pressure threshold;
the first and second pressure thresholds being associated with respective fluid pressures to enable a portion of an expiration phase by a human and directed into the inlet valve to be captured within the internal reservoir.

10. The pressure actuated fluid capturing device as recited in claim 9, wherein the body includes:
a central body;
a first end body engageable with the central body via snap-fit engagement; and
a second end body engageable with the central body via snap-fit engagement.

11. The pressure actuated fluid capturing device as recited in claim 10, further comprising a first gasket extending between the central body and the first end body to create a fluid tight seal therebetween, and a second gasket extending between the central body and the second end body to create a fluid tight seal therebetween.

12. The pressure actuated fluid capturing device as recited in claim 10, further comprising a bladder coupled to the first end body and the second end body, the bladder at least partially defining the internal reservoir and being adapted to contact a prescribed surface on the body when a prescribed volume of fluid is received within the bladder, the bladder and the body being configured such that the contact between the bladder and the prescribed surface on the body provides a visual indication to a user.

13. The pressure actuated fluid capturing device as recited in claim 10, wherein the first end body includes a mouthpiece adapted to enable a mouth of a user to breath into the device through the inlet valve.

14. The pressure actuated fluid capturing device as recited in claim 9, further comprising an extraction valve coupled to the body and in fluid communication with the internal reservoir, the extraction valve being separate from the inlet valve and the outlet valve.

15. A method of capturing a portion of a user's breath, the method comprising:
receiving breath exhaled from a user in a breath capturing device;
capturing a first portion of the breath exhaled by the user within the breath capturing device such that the fluid pressure within the breath capturing device increases;
venting at least a portion of the captured breath from the breath capturing device when the fluid pressure within the breath capturing device exceeds a prescribed pressure threshold; and
capturing a second portion of the breath exhaled by the user and subsequent to the venting step in response to the fluid pressure within the breath capturing device falling below the prescribed pressure threshold.

16. The method recited in claim 15, wherein all steps are performed during a continuous exhalation phase by the user.

17. The method recited in claim 15, wherein the first and second portions of the breath exhaled by the user during a continuous exhalation phase.

18. The method recited in claim 15, wherein the breath capturing device includes an exhaust port which remains closed while capturing the first portion of the breath exhaled by the user, and which opens during the venting step.

19. The method recited in claim 15, wherein the venting step includes transitioning a vent valve from a closed position to an open position in response to fluid pressure within the breath capturing device exceeding a prescribed vent threshold.

20. The method recited in claim 19, wherein the step of capturing the second portion of the breath includes transitioning the vent valve from the open position to the closed position in response to the fluid pressure within the breath capturing device falling below the prescribed vent threshold.
